(19) United States
(12) Patent Application Publication
(10) Pub. No.: US 2020/0100901 A1
(43) Pub. Date: Apr. 2, 2020

(54) BONE CEMENT MOLD

(71) Applicant: IDEALFIT SPACER TECHNOLOGIES INC., London (CA)

(72) Inventors: Brent A. LANTING, London, Ontario (CA); Matthew G. TEETER, London, Ontario (CA); Hristo NIKOLOV, London, Ontario (CA); David W. HOLDSWORTH, London, Ontario (CA); Mohammad Ali TAVALLAEI, London, Ontario (CA); John MATHESON, London, Ontario (CA); Asha PAREKH, London, Ontario (CA)

(21) Appl. No.: 16/622,246

(22) PCT Filed: Jun. 16, 2017
(86) PCT No.: PCT/CA2017/050745

§ 371 Date: Dec. 12, 2019

(51) Int. Cl. A61F 2/30; A61F 2/38

(52) U.S. Cl. CPC A61F 2/30942; A61F 2/30724; A61F 2/3859; A61F 2/389; A61F 2002/4633; A61F 2002/3069; A61F 2002/30677; A61F 2002/30957; A61F 2/3094

(57) ABSTRACT

A bone cement mold for a bone cement spacer, the bone cement mold including: a first mold component defining a first cavity shaped to form at least a majority of a head portion of the spacer; a second mold component defining a second cavity shaped to form a first part of a stem portion of the spacer; a third mold component defining a third cavity shaped to form a second part of the stem portion of the spacer; the second mold component and the third mold component combining to define a cavity shaped to form at least a majority of the stem portion; an abutting combination of the first mold component, second mold component, and third component defining a communicative cavity shaped to form the head portion integrally connected with the stem portion. Bone cement spacers formed from the bone cement mold, and computer methods and systems for selecting and operating the bone cement mold are also described.

### BACKGROUND OF THE INVENTION

### Field of the Invention

The present invention relates to joint replacement surgeries and in particular, revision surgeries that are required when an implanted joint prosthesis must be removed.

### Description of the Related Art

Infection following a joint replacement surgery occurs in a significant number of patients. Periprosthetic joint infection is considered the most challenging complication of joint replacement with perhaps the largest associated cost of any type of revision surgery. Due to an aging population and increased obesity rates (which result in younger patients needing joint replacement) increasing numbers of hip replacements and knee replacements are expected. Accordingly, joint replacement surgeries and their associated risks of infection and revision surgery place a significant burden on physicians, hospitals and taxpayers.

For example, infection following total knee arthroplasty (TKA) occurs in approximately 2% of cases. This numbers approximately 14,000 cases per year and rising in the United States, and is associated with a significant cost projected to be $1.6 billion USD in 2020.

An accepted standard for treatment of TKA infection is a two stage procedure: in stage one, the infected joint prosthesis is removed and replaced with an antibiotic-impregnated bone cement spacer, left in place for weeks to months while the infection clears; in stage two, the spacer is removed and replaced with a new revision joint prosthesis.

Cement spacers can function to deliver therapeutic compounds, maintain joint spacing, maintain tissue integrity, minimize soft-tissue loss, some range of motion, some degree of ambulation or any combination thereof.

Currently utilized cement spacers can be purchased as preformed units, or can be made intraoperatively using molds; with the latter method advantageously allowing a surgeon to choose a suitable bone cement and an effective antibacterial or antifungal agent (as well as effective dosage/concentration) to specifically target the infectious organism(s) found in the patient.

Cement spacers that are currently utilized have a number of known complications, including debonding, spacer fracture, and knee instability, which all relate to poor fit and/or fixation of the spacer within the bone.

Accordingly, there is a continuing need for alternative bone cement spacers and molds to produce bone cement spacers.

### SUMMARY OF THE INVENTION

In an aspect there is provided, a bone cement mold for a bone cement spacer, the bone cement mold comprising:

a first mold component defining a first cavity shaped to form at least a majority of a head portion of the spacer;

a second mold component defining a second cavity shaped to form a first part of a stem portion of the spacer;

a third mold component defining a third cavity shaped to form a second part of the stem portion of the spacer;

the second mold component and the third mold component combining to define a cavity shaped to form at least a majority of the stem portion;

an abutting combination of the first mold component, second mold component, and third component defining a communicative cavity shaped to form the head portion integrally connected with the stem portion.

In other aspects, bone cement spacers formed from the bone cement mold, and computer methods and systems for selecting and operating the bone cement mold are also provided.

### BRIEF DESCRIPTION OF THE DRAWINGS

FIG. 1 shows several views of a tibial spacer;

FIG. 2 shows several views of a bone cement mold useful to form the tibial spacer shown in FIG. 1;

FIG. 3 shows several views of a femoral spacer;

FIG. 4 shows several views of a bone cement mold useful to form the femoral spacer shown in FIG. 3;

FIG. 5 illustrates operation of a modified version of the tibial spacer shown in FIG. 1;

FIG. 6 shows (A) a first modified version of the tibial spacer shown in FIG. 1; and (B) a first modified version of the femoral spacer shown in FIG. 3;

FIG. 7 shows (A) a second modified version of the tibial spacer shown in FIG. 1; and (B) a second modified version of the femoral spacer shown in FIG. 3;

FIG. 8 shows (A) a third modified version of the tibial spacer shown in FIG. 1; and (B) a third modified version of the femoral spacer shown in FIG. 3;

FIG. 9 shows (A) a fourth modified version of the tibial spacer shown in FIG. 1; and (B) a fourth modified version of the femoral spacer shown in FIG. 3;

FIG. 10 shows (A) an endoskeleton; and (B) an operable insertion of the endoskeleton within a modified version (basic stem, no augment) of the tibial bone cement mold shown in FIG. 2;

FIG. 11 shows a flowchart illustrating a computer-implemented method for selecting a bone cement mold matched to a user request;

FIG. 12 shows a flowchart providing greater detail of steps shown in FIG. 10;

FIG. 13 shows a flowchart providing greater detail of steps shown in FIG. 10.

FIG. 14 shows a flowchart illustrating the steps of FIG. 11 in a larger sequence of steps for creation of a customized orthopaedic joint spacer;

FIG. 15 shows a schematic diagram of a computer system configured to implement the method shown in FIG. 11 or 14;

FIG. 16 shows an example of an interactive graphical user interface that may be displayed and used to input parameters from a user computer device shown in FIG. 15.

### DETAILED DESCRIPTION OF PREFERRED EMBODIMENTS

Teachings provided herein relate to bone cement spacers, molds and computer-implemented methods for producing the same, that find use in the field of joint replacement surgery. Spacers are temporary implants that are intended to be maintained in a joint on a time span of weeks to months, and therefore the terms spacer and temporary implant may be used interchangeably. Joint prosthetics are permanent implants or primary implants that are intended to be maintained in a joint on a time span of years, and therefore the terms prosthetics and permanent implants and primary implants may be used interchangeably.

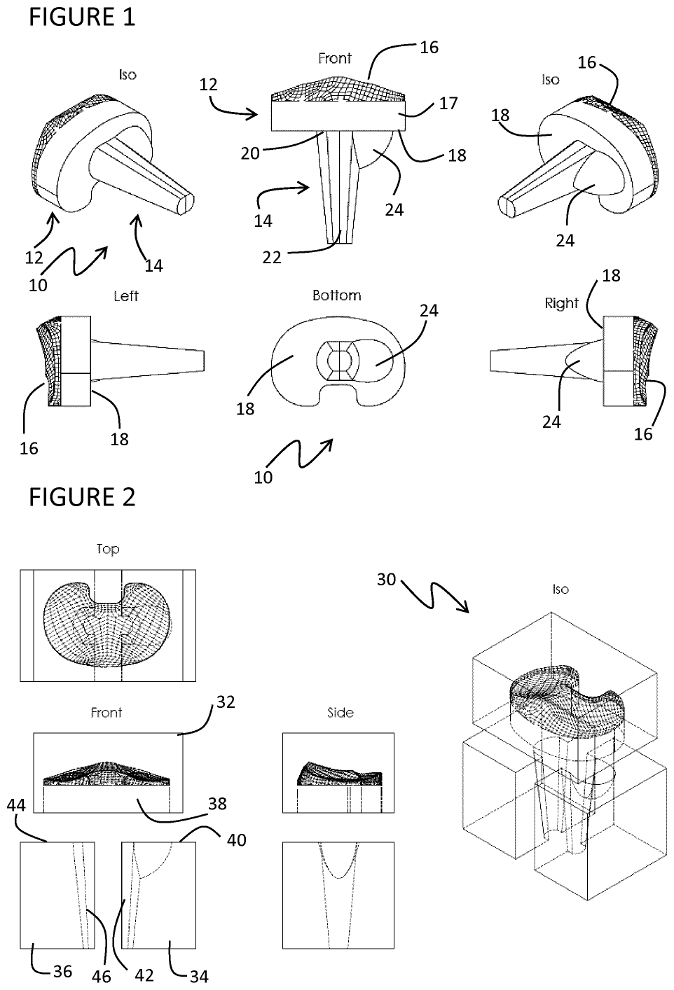

Referring to FIGS. 1 and 2, a tibial spacer and its corresponding mold are described, respectively. The tibial spacer 10 comprises a head portion 12 for abutting and capping a condyle region of the tibia and a stem portion 14 for insertion into the medullary canal of the tibia. The head portion 12 is bound by an articulating surface 16, a bone-facing surface 18, and a sidewall 17 co-extensively joining the articulating surface 16 to the bone-facing surface 18 at their corresponding periphery. The stem portion 14 is an elongate columnar structure having a conical shape extending from a base end 20 to a tip end 22; the base end has a larger diameter than the tip end. The base end 20 connects to and is integrally formed with the bone-facing surface 18 of the head portion 12. The tip end 22 is a free end that does not connect with any structure and leads insertion of the tibial spacer 10 into the tibial medullary canal. The stem portion 14 includes an augmented surface feature 24 at the base end 20. The augmented surface feature 24 is a rounded dome-shaped projection or protuberance that extends radially outward at or near the base end 20.

FIG. 2 shows a three component mold used to form the tibial spacer 10. The three component mold 30 comprises a first component 32 formed with a cavity defining the articulating surface 16 of the head portion 12 of the tibial spacer 10; and a second component 34 and a third component 36 that each form cavities that combine to define the stem portion 14 and the bone-facing surface 18 of the head portion 12. The first component 32 is formed with a head-shaped cavity 38 shaped and sized to define the articulating surface 16 and the sidewall 17 of the head portion 12. The second component 34 provides a first planar surface 40 to define a part of the bone-facing surface 18 of the head portion 12 and a first partial-stem-shaped cavity 42 shaped and sized to define a longitudinal cut of the stem portion that includes augmented surface feature 24; the first planar surface 40 is oriented substantially perpendicular to the longitudinal dimension of the first partial-stem-shaped cavity 42. The third component 36 provides a second planar surface 44 to define a part of the bone-facing surface 18 of the head portion 12 and a second partial-stem-shaped cavity 46 shaped and sized to define a longitudinal cut of the stem portion; the second planar surface 44 is oriented substantially perpendicular to the longitudinal dimension of the second partial-stem-shaped cavity 46. The first planar surface 40 and the second planar surface 44 combine to define the bone-facing surface 18, while the first partial-stem-shaped cavity 42 and the second partial-stem-shaped cavity 46 combine to form the entire stem portion 14.

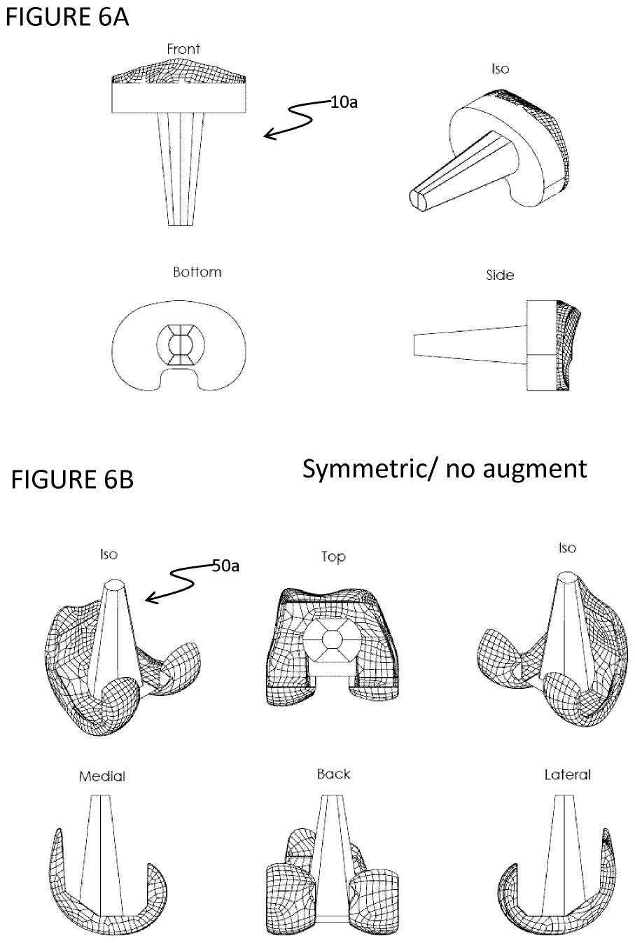

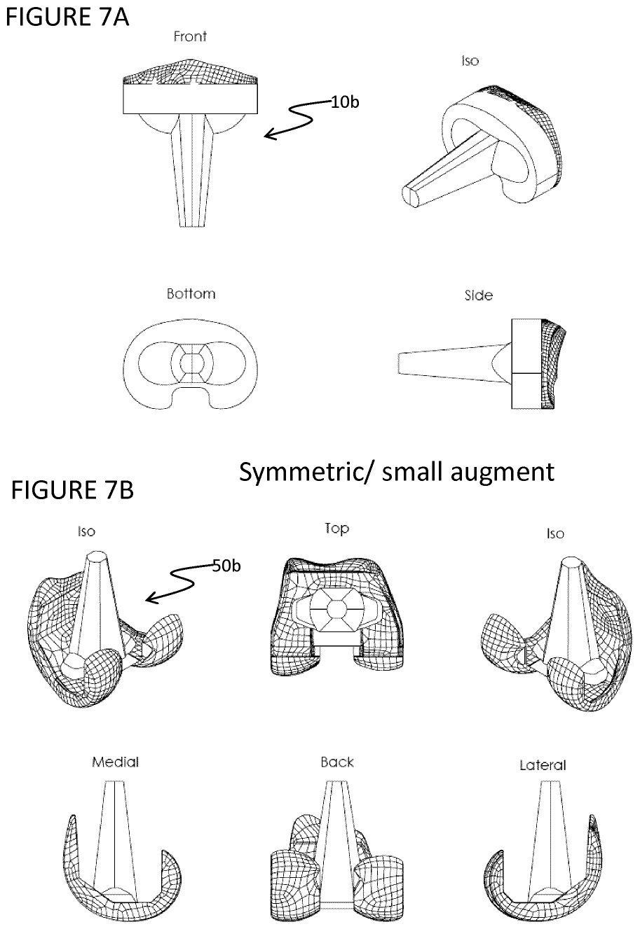

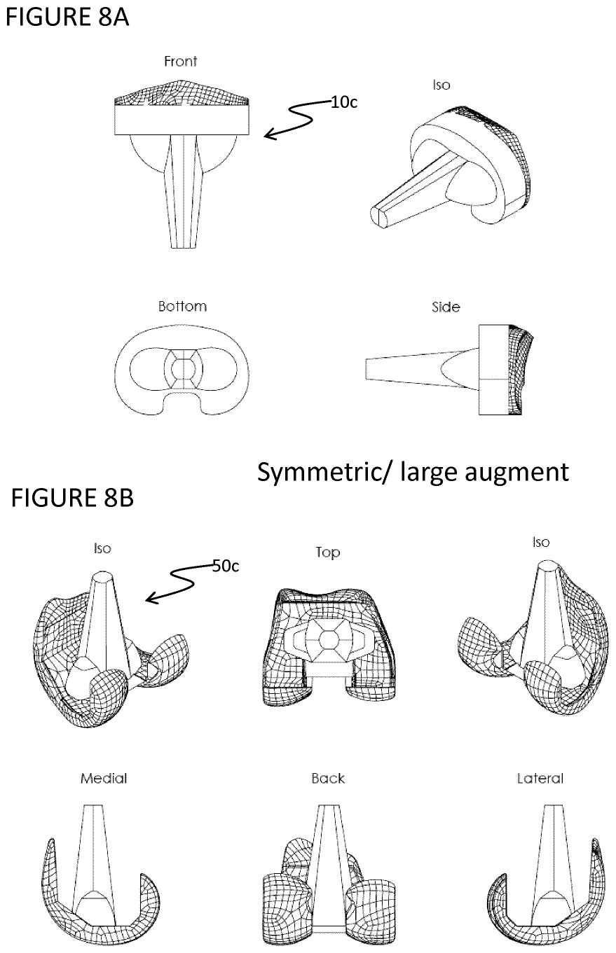

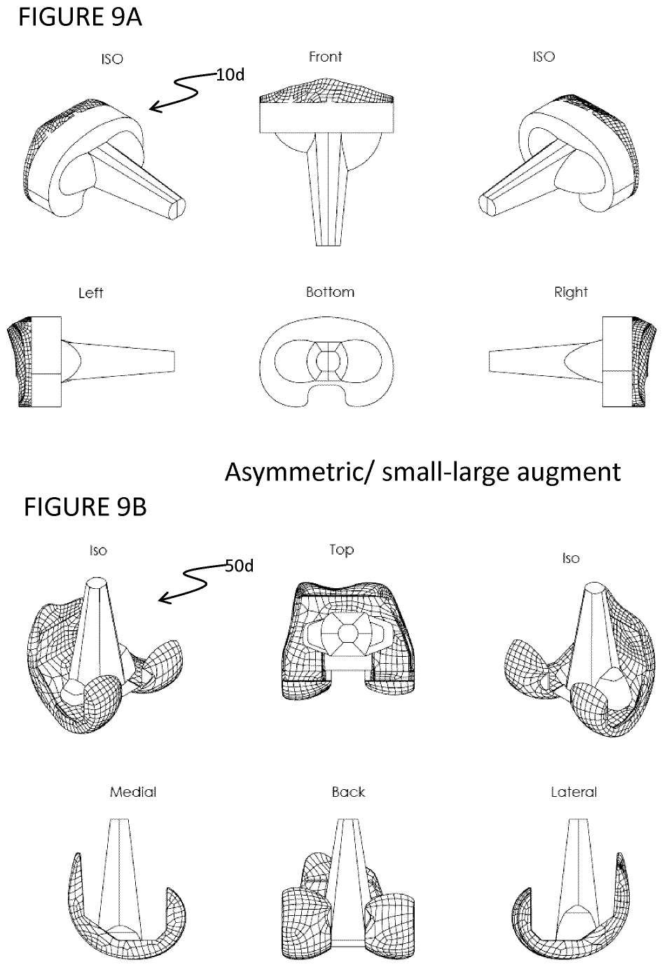

The cavity and surface shapes of the first, second and third components may be modified to form a tibial spacer of a desired shape or dimension. For example, the size and/or shape of the stem portion may be modified by forming cavities of the second and/or third components to a desired shape. Similarly, the shape and position of augmented surface feature 24 may be modified as desired. Similarly, the first planar surface 40 and the second planar surface 44 may be independently substituted with a shaped cavity to define any desired surface shape or surface feature for the bone-facing surface of the tibial spacer. Similarly, the thickness of the head portion may be readily adjusted by adjusting the depth of the head-shaped cavity 38 formed in the first component 32. Similarly, the shape and position of augmented surface feature 24 may be modified as desired. Examples of variations of size, position and number of augmented surface feature 24 formed using the three component mold for a tibial spacer are shown in FIG. 6A (tibial spacer 10a having symmetrical stem with no augmentation present), FIG. 7A (tibial spacer 10b having symmetrical stem with equally sized small augmentation on both medial and lateral sides), FIG. 8A (tibial spacer 10c having symmetrical stem with equally sized large augmentation on both medial and lateral sides), and FIG. 9A (tibial spacer 10d having asymmetrical stem with differently sized augmentation on medial side compared to lateral side).

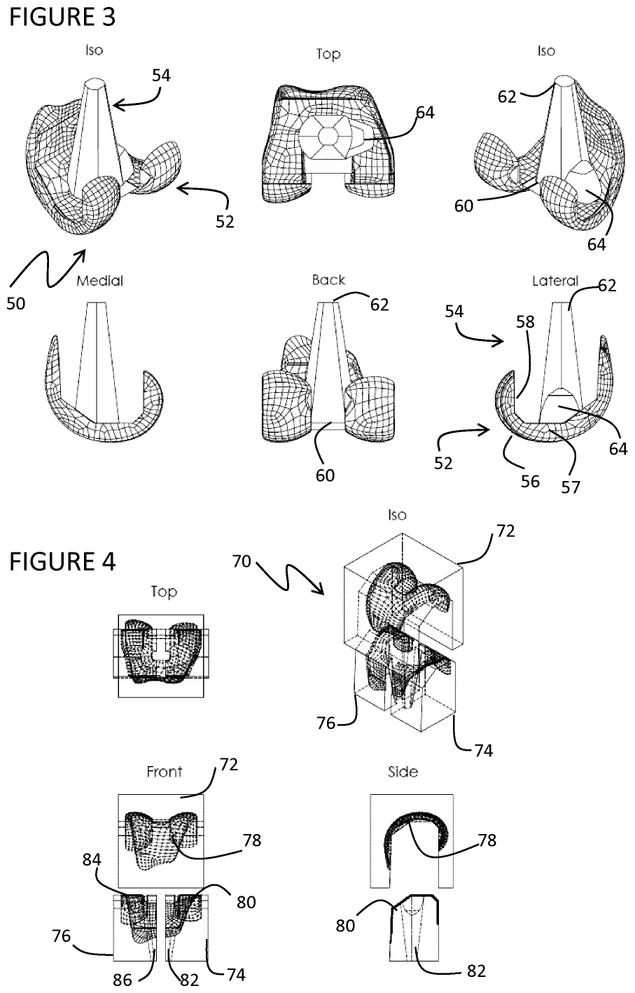

Referring to FIGS. 3 and 4, a femoral spacer and its corresponding mold are described, respectively. The femoral spacer 50 comprises a head portion 52 for abutting and capping a condyle region of the femur at the knee joint and a stem portion 54 for insertion into the medullary canal of the femur end at the knee joint. The head portion 52 is bound by an articulating surface 56, a bone-facing surface 58, and a sidewall 57 co-extensively joining the articulating surface 56 to the bone-facing surface 58 at their corresponding periphery. The head portion 52 is U-shaped with the articulating surface 56 analogous to the outer convex surface of the U-shape and the bone-facing surface 58 analogous to the inner concave surface of the U-shape. The stem portion 54 is an elongate columnar structure having a conical shape extending from a base end 60 to a tip end 62; the base end has a larger diameter than the tip end. The base end 60 connects to and is integrally formed with the bone-facing surface 58 of the head portion 52. The tip end 62 is a free end that does not connect with any structure and leads insertion of the femoral spacer 50 into the femur medullary canal. The stem portion 54 includes an augmented surface feature 64 at the base end 60. The augmented surface feature 64 is a rounded dome-shaped projection or protuberance that extends radially outward at or near the base end 60.

FIG. 4 shows a three component mold used to form the femoral spacer 50. The three component mold 70 comprises a first component 72 formed with a cavity defining the articulating surface 56 of the head portion 52 of the femoral spacer 50; and a second component 74 and a third component 76 that each form cavities that combine to define the stem portion 54 and the bone-facing surface 58 of the head portion 52. The first component 72 is formed with a head-shaped cavity 78 shaped and sized to define the articulating surface 56 and the sidewall 57 of the head portion 52. The second component 34 provides a first partial bone-facing-surface-shaped cavity 80 to define a part of the bone-facing surface 58 of the head portion 52 and a first partial-stem-shaped cavity 82 shaped and sized to define a longitudinal cut of the stem portion that includes augmented surface feature 64; the first partial bone-facing-surface-shaped cavity 80 is U-shaped, with the base of the U-shape oriented substantially perpendicular to the longitudinal dimension of the first partial-stem-shaped cavity 82. The third component 76 provides a second partial bone-facing-surface-shaped cavity 84 to define a second part of the bone-facing surface 58 of the head portion 52 and a second partial-stem-shaped cavity 86 shaped and sized to define a longitudinal cut of the stem portion; the second partial bone-facing-surface-shaped cavity 84 is U-shaped, with the base of the U-shape oriented substantially perpendicular to the longitudinal dimension of the second partial-stem-shaped cavity 86. The first partial bone-facing-surface-shaped cavity 80 and the second partial bone-facing-surface-shaped cavity 84 combine to define the bone-facing surface 58, while the first partial-stem-shaped cavity 82 and the second partial-stem-shaped cavity 86 combine to form the entire stem portion 54.

The cavity and surface shapes of the first, second and third components may be modified to form a femoral spacer of a desired shape or dimension. For example, the size and/or shape of the stem portion may be modified by forming cavities of the second and/or third components to a desired shape. Similarly, the thickness of the head portion may be readily adjusted by adjusting the depth of the head-shaped cavity 78 formed in the first component 72; while adjustment of thickness is readily achievable, in practice adjustment of thickness of the femoral head portion is typically not needed, as thickness adjustments are typically implemented in the tibial head portion. Similarly, the shape and position of augmented surface feature 64 may be modified as desired. Examples of variations of size, position and number of augmented surface feature 64 formed using the three component mold for a femoral spacer are shown in FIG. 6B (femoral spacer 50a having symmetrical stem with no augmentation present), FIG. 7B (femoral spacer 50b having symmetrical stem with equally sized small augmentation on both medial and lateral sides), FIG. 8B (femoral spacer 50c having symmetrical stem with equally sized large augmentation on both medial and lateral sides), and FIG. 9B (femoral spacer 50d having asymmetrical stem with differently sized augmentation on medial side compared to lateral side).

An example of operation of the three component mold to produce a spacer is use by a surgeon or technician for in situ production of a spacer for revision surgery to treat a periprosthetic infection subsequent to a joint replacement surgery. The desired bone cement is mixed with an effective type and amount of antibiotic which is then mixed with a liquid monomer to initiate a polymerization process. The bone cement may be any suitable biocompatible cement such as a poly-methyl-methacrylate (PMMA) cement, or a MMA-styrene copolymer, or an MMA-methyl acrylate copolymer. The antibiotic may be tobramycin or gentamicin or any other suitably effective antibiotic. Once the appropriate antibiotic loaded bone cement is mixed, the bone cement is loaded into a delivery device such as a gun or a syringe which is then used to load mixed bone cement into the three component mold. Once the cement is cured the mold is removed to produce the spacer for use in a revision surgery.

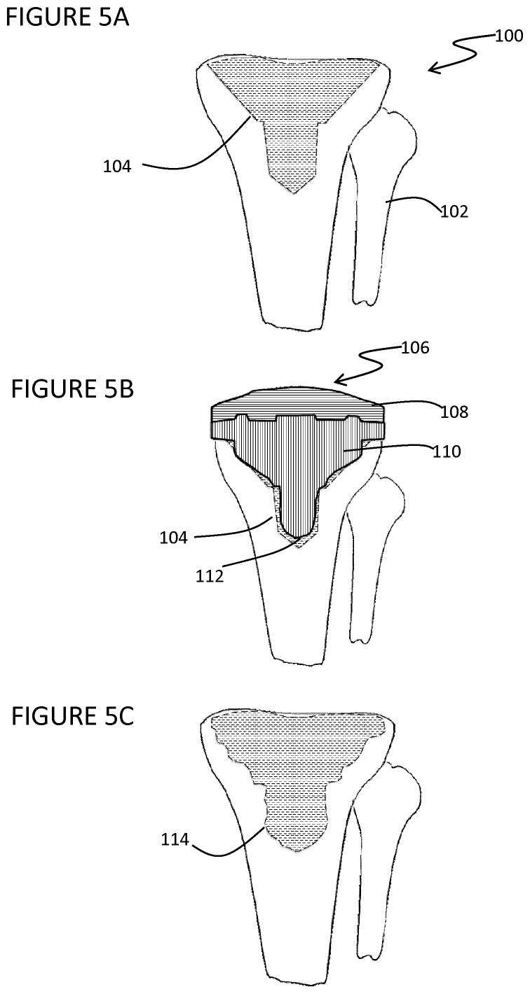

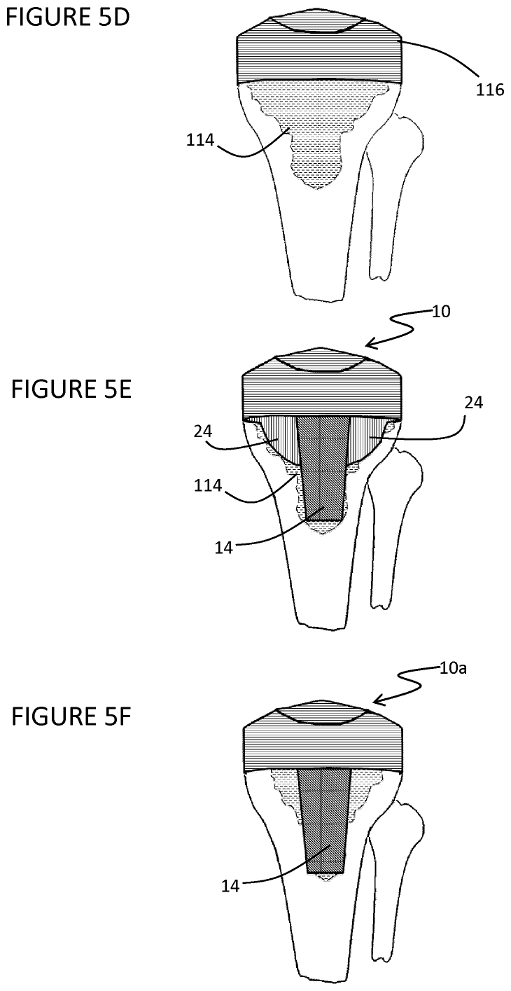

FIG. 5 (5A to 5F) illustrates steps of a revision surgery demonstrating benefits of spacers produced according teachings provided herein as compared to conventional spacers. FIG. 5A shows a tibia end 100 prepared for insertion of a prosthetic in a keen joint replacement surgery. A fibula 102 is shown simply for anatomical context and need not be further described. For a primary knee joint replacement surgery, the proximal tibia is resected as desired to form bone cuts 104 and the intramedullary canal is broached so as to fit a tibial knee joint prosthetic 106 (FIG. 5B). The tibial knee joint prosthetic comprises a tibial insert 108 that comprises an articulating surface and a tibial component 110 that fits within the intramedullary canal. The implanted tibial component 110 substantially fills the space defined by bone cuts 104, with bone cement 112 applied between the surfaces of the tibial component 110 and the surfaces of the bone cuts 104 to ensure a stable and rigorous attachment (the attachment is intended to be permanent).

If a persistent infection arises as a complication of the primary knee joint replacement surgery, for example a periprosthetic infection, a revision surgery may be needed. A standard remedy for infection is a two-stage revision surgery comprising: 1) removal of the initial implant, followed by debridement of the infected tissue; 2) insertion of an antibiotic impregnated cement spacer; and after a typical time interval of weeks to months 3) removal of the cement spacer once the infection has been eradicated and insertion of a new implant. In addition to antibiotic delivery, cement spacers may be used to maintain joint spacing, minimize soft-tissue loss, preserve range of motion, and/or provide some degree of ambulation if the spacers are articulating.

Steps 1 and 2 are the first stage of the two-stage revision surgery and step 3 is the second stage. FIG. 5C shows step 1 of the first stage of the two-stage revision surgery of revision subsequent to removal of the tibial knee joint prosthetic 106 and any needed debridement procedure often resulting in an enlarged space with uneven bone defects 114 due to tibial component 110 removal as compared to the more regular working surfaces of bone cuts 104.

Step 2 involves producing a suitably shaped cement spacer to adequately fit bone defects 114. In order to produce a suitably shaped cement spacer the model/size of the primary prosthetic can be provided and optionally the presence of any bone defects that need to be addressed (as imaged, for example, by x-ray). Computer software can be used to select/determine 3D geometry based on the model/size of the primary prosthetic (ie., 3D geometry of primary prosthetic obtained through any convenient source including, for example, laser scanning, micro-CT, manufacturer CAD drawings), and the selected 3D geometry can then be optionally adjusted to account for observed bone defects. Once the 3D geometry is established, computer software may execute a matching algorithm to select an optimized combination of a three component mold from a pre-existing library containing a plurality of categorized or annotated first, second and third components defining an array of assorted shapes and sizes of head portions, stem portions and augmented surface features. Accordingly, the three component mold selected from a preset library can be quickly and conveniently assembled as compared to a de novo configuration of a mold based on imaged geometry of prosthetic and bone defects. Once the suitable three component mold is identified by computer software, the specific physical components may be assembled to be loaded with bone cement mixed with an effective type and amount of antibiotic targeting a patient's infectious organism.

As shown in FIG. 5D conventional tibial spacers do not include stems, and therefore a conventional spacer 116 can only be cemented to the top of the proximal tibia, and do not fill the bone defects 114, resulting in poor fixation. In comparison, FIG. 5E shows that a tibial spacer 10 as described herein provides a better fit for bone defect surfaces due to removal of prosthetic 106; the tibial spacer 10 comprises a stem 14 with augmented surface features 24 at the base of the stem, the stem and the augmentations combining to suitably fill the bone defects 114 for improved fixation and stability. As shown in FIG. 5F, when bone defects are not prominent augmented surface features extending from the stem may be smaller or unnecessary, but a stem is nevertheless useful to enhance fixation and stability. Furthermore, additional bone cement with an appropriate antibiotic/antifungal may be used to cement each stem into the femur and tibia to further enhance strength of fit of the spacer.

The better fit obtained from the spacers provided in accordance with the present teachings will avoid recognized complications (e.g. dislocations, subluxations). Moreover, the spacers described herein provide the surgeon with the ability to customize the antibiotic and tailor the concentrations to the patient's infection needs.

Several advantages of using the three component mold described herein to produce spacers become apparent when comparing spacers produced by the three component mold to conventional tibial or femoral spacers.

Spacers that are currently utilized have a number of known complications, including debonding, spacer fracture, and knee instability, which all relate to poor fit and/or fixation of the spacer within the bone. An example of an advantage of the three component mold to form spacers is that the stem in a spacer provides (A) improved stabilization and fit (femoral and tibial spacers with a stem provide stronger intramedullary fixation, to resist debonding) and (B) improved drug delivery to bone surfaces such as intramedullary delivery of therapeutic drugs (ie., delivery of antibiotics or other suitable therapeutic compounds (anti-fungal or anti-inflammatory compounds) to the medullary cavity of a tibia and/or femur).

Another example of an advantage of the three component mold is that the spacer size and shape, and particularly the stem portion size and shape, can be prosthetic specific based on known dimensions/shape/geometry of the prosthetic (ie., permanent implant) being removed for revision surgery; furthermore, the spacer size and shape may be patient specific to compensate for bone defects after implant removal (based for example by viewing x-ray image of bone cuts and/or bone defects shape after removal of the prosthetic).

Another example of an advantage of the three component mold is an integral formation of stem portion with a head portion of a spacer for greater strength and stability of the spacer.

Another example of an advantage of the three component mold is that it permits ease of insertion of endoskeleton.

Several illustrative variants have been described above. Further variants and modifications are described below. Moreover, guiding relationships for configuring variants and modifications are also described below. Still further variants and modifications are contemplated and will be recognized by the person of skill in the art. It is to be understood that guiding relationships and illustrative variants or modifications are provided for the purpose of enhancing the understanding of the person of skill in the art and are not intended as limiting statements.

First, second and third components of the three component mold may be fastened together using any conventional fastener. For example, any convenient male/female fastener may be operable including, for example, tongue and groove or bore and bolt fasteners. As an alternative example, clips or clamps may be used to hold the components together. As another example, components may be reversibly fastened together using a reversible adhesive. As yet another alternative example, components may be operably attached using a permanent adhesive and subsequently detached using specialized cutting tools after curing is complete.

Any suitable bone cement and therapeutic compound combination may be loaded into the three component mold. The three component mold may have an inlet operable for loading of a bone cement mix that is ready to be polymerized. For example, the tip of the stem being a free end permits a convenient inlet for loading of bone cement; the second and third mold components that combine to form the stem can define an opening at a location corresponding to the tip end of the stem, allowing cement to be introduced through the opening. Alternatively, one or more specialized inlet port(s) may be incorporated as desired. Inlets may be configured as suited to a corresponding bone cement delivery device such as a syringe or gun so as to provide operable fluid communication between the inlet of the mold component(s) and the outlet of the corresponding bone cement delivery device or applicator.

Bone cement may be loaded into a three component mold using any convenient delivery device or applicator tool including, for example, a syringe, a gun, a tongue depressor, a spatula, and the like.

The three component mold may include ventilation apertures that allow for escape of air during loading of bone cement and allow for easy evaporation for a curing step.

The three component mold may include any convenient handle, grip or texturized surface feature operable to ease or guide manual manipulation of one or more of the three components. For example, each of the components may be formed with an integrated grip or tab for ease of assembling the three components prior to loading of a bone cement mix and/or disassembly of the three components after the bone cement mix has set and cured.

The three component mold may include an endoskeleton. The endoskeleton may be positioned within an assembled three component mold using any convenient connector. When an endoskeleton is inserted within the three component mold, the endoskeleton is incorporated within the cured bone cement spacer. The endoskeleton may be linear or branched as desired for a specific application. The endoskeleton may be constructed of any suitable medical grade metal or polymer (for example, polyether ether ketone—PEEK) The endoskeleton typically comprises an elongate body configured to span the stem and head portions and to fit within the designed geometry of the cavity defined by the three component mold, so that it does not extend beyond the boundaries of the head portion, particularly the articular surface. The long axis of the endoskeleton can be substantially co-aligned with the long axis of the cavity defining the stem portion. The endoskeleton may be designed with a temporary stabilizer end, such as a T-shaped or X-shaped hook with the arms of the T-shape or X-shape extending perpendicular to the long axis of the endoskeleton. The stabilizer end may abut or couple to or engage in any suitable manner an outer surface of an opening communicative with the end of the stem-defining cavity corresponding to the tip end of the stem. As the stabilizer end is held at or within the opening of the stem-defining cavity, the elongate body of the endoskeleton is held in proper position substantially along the long axis of the stem extending into the head portion without piercing or extending beyond the outer surface of the head. After the cement is introduced and cured, the temporary stabilizer end can be clipped off at the tip of the stem using any suitable surgical tool. If the clipped endoskeleton end is exposed at the tip end of the stem—a small dab of cement can be used to cover it.

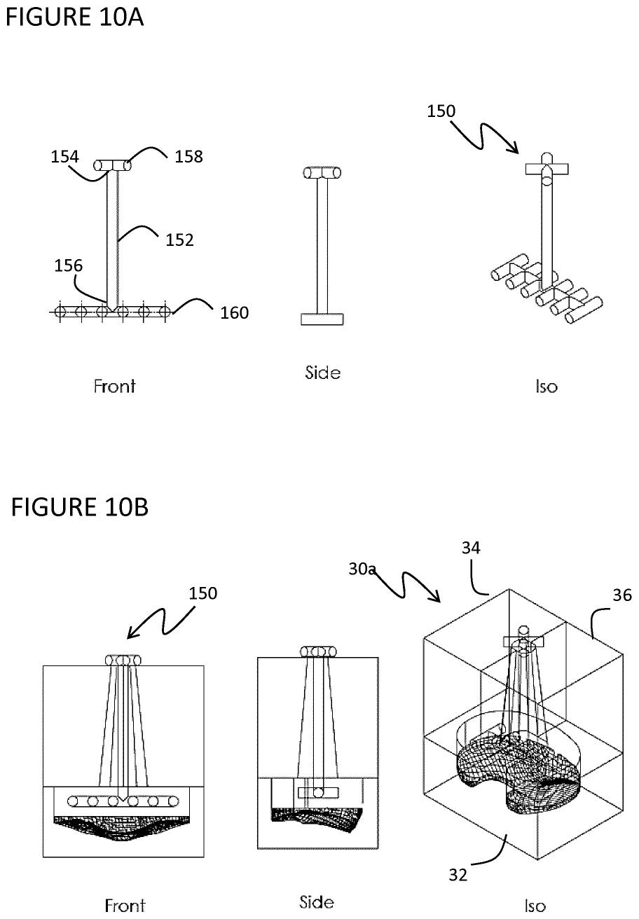

FIG. 10A shows an example of an endoskeleton 150. The endoskeleton 150 comprises an elongate body 152 having a stabilizer end 154 and a base end 156. An X-shaped hook/hanger 158 extends radially from the stabilizer end 154 with the intersection of the X co-axially aligned with the longitudinal axis of the elongate body 152. An endoskeleton head portion 160 extends radially from the base end 156 with the head portion 160 aligned substantially transverse to the longitudinal axis of the elongate body 152.

FIG. 10B shows an example of the endoskeleton 150 operably inserted within a modified version (assembled mold forms a cavity defining a basic stem shape without any augmented surface feature) of the tibial bone cement mold shown in FIG. 2. The endoskeleton 150 is inserted within tibial bone cement mold 30a. The X-arms of hook/hanger 158 abut surfaces surrounding the opening defining the tip end of the stem of the spacer, allowing a desired position of the endoskeleton 150 to be maintained within bone cement mold 30a. Any convenient stabilizer element may be disposed at the stabilizer end 154. For example, hook/hanger 158 may be replaced with a conical cork/stopper that wedges (interference friction fit) within the opening defining the tip end of the stem.

While the integrally formed stem confers stabilization and fixation benefits to that spacer, the incorporation of an endoskeleton can further improve stability and robustness/strength of the spacer. Thus, a bone cement spacer comprising a stem portion integrally connected to a head portion and an endoskeleton contained within the bone cement spacer may provide advantages heretofor unrecognized in joint revision surgical procedures. Typically, the endoskeleton will extend from the stem portion to the head portion to reinforce the integral connection of the stem to the head. In certain examples, the endoskeleton spans a full longitudinal dimension of the stem portion. In another example, the endoskeleton comprises an elongate body and a transverse end portion sized to be smaller than the head portion of the spacer. The transverse end may comprise any convenient shape to conform to the shape of the head portion of the spacer, including shapes of triangles, discs, spheres, rings, domes, U-shapes, and the like. In yet another example, the longitudinal axis of the elongate body is substantially aligned with the longitudinal axis of the stem portion.

The three component mold may be applicable to both articulating and static spacers. Articulating spacers are shown in the drawings (for example, see FIGS. 1 to 4). Where static spacers are desired, the articulating surface may be designated as a joint-cavity-facing surface and may be shaped as desired, often having a more planar shape compared to an articulating surface. The three component mold include a first component defining a majority of the volume of head portion of a spacer, while second and third components will operably combine to define the shape and volume of a majority of the stem portion, and typically the entire stem portion. The second and third components may be described as left/right components for illustrative purposes using conventional directional terms. Alternatively, the second and third components may be described as medial/lateral components for illustrative purposes using conventional anatomical reference. However, the second and third components may also use other anatomical geometries, for example dorsal/ventral, dorsomedial/ventrolateral, ventromedial/dorsolateral. More generally, the three component mold comprises a first component that defines at least a majority of the volume of the head portion of the spacer, and second and third components that together form a cavity defining a stem portion of the spacer and also define a bone-facing surface of the head portion. The second and third components each provide an interior surface that defines a part of the radial dimension of stem while defining the full axial dimension of the stem, so that each of the second and third components form a cavity to define a pair of complimentary pieces of the stem cut longitudinally, and an abutting combination of the second and third components combine together to form a cavity defining an entire stem. The pair of complimentary pieces of the stem will often be cut radially so that each of the second and third components define a substantially equal length of circumference or substantially equal volume of the stem (when not considering augmented surface features for convenience of considering this example). However, the pair of complimentary pieces of the stem need not be substantially equal and the second and third components may relatively define greater or lesser amounts of the stem provided that together the second and third components combine to define substantially the entire stem. For example, the second component may define two-thirds of the circumference of the stem if the third component defines the remaining one-third of the circumference.

Two-stage revision surgery involves removal of a first permanent implant, and installation of a temporary implant in a first stage, and then after a suitable time interval, removal of the temporary implant and installation of a second permanent implant in a second stage. The term implant may then be generally used for both permanent and temporary implant. For clarity, the term prosthesis may be used to reference a permanent implant, while the term spacer may be used to reference a temporary implant. Spacers described herein include bone cement spacers, and spacers incorporating therapeutic compounds such as antibiotic-impregnated bone cement spacers.

The three component mold can be consolidated as a two component mold wherein the first component defines a medial portion of the articulating and stem portions of the spacer and the second component defines a lateral portion of the articulating and stem portions of the spacer so that the two components together form a cavity defining the spacer. However, such a two component mold suffers distinct disadvantages when it comes to flexibility of altering shape and size of the stem or head portion of the spacer. The three component mold can be provided as multiple versions of each of the three components that can be mixed/matched and combined as desired to define a suitable stem and head portions for a specific application. The multiple versions of each of the three components may be collected as objects in a set, (such as a set of differing augmented surface features) and categorized according to any desired criteria (such as make/model of prosthetic or size of augmented surface feature) to allow for manual or computer automated matching of a single version/object from each set to produce a matched three component mold for a specific application. For convenience sets may be distinguished by geometry or shape with the individual objects of each set distinguished by size intervals; however, many other categorization schemes may be applicable. In certain applications, each of the three components may be provided as multiple sets with a plurality of versions in each set, with the collection of the multiple sets categorized and annotated in a physical and/or digital library.

Kits may be developed that include the three component mold and instructions for its use to produce a spacer. Kits may additionally include one or more of bone cement powder, polymerization initiator, endoskeleton, bone cement applicator such as a syringe or gun. Kits may be developed with a set of a plurality of versions of one or more of the components of the three component mold with tables for assisting selection of one version from the set.

Bone cement spacers will often be impregnated with a desired antibiotic, but the spacer is not limited to delivery of antibiotics and could deliver other therapeutic compounds, such as antifungal or anti-inflammatory compounds.

While molds for bone cement spacers are described for knee revision procedures, molds described herein can readily be adapted to revision procedures for other joints, for example hip joint revision procedures or shoulder joint revision procedures.

Molds may be manufactured using any convenient technique. For example, direct production using 3D printing technology or numerically controlled milling machines. Another example, is to produce a desired shape of the spacer and form a mold around it. Molds may be further processed as desired using any number of known techniques such as milling, sanding or sandblasting.

Any suitable material may be used to construct a mold including a metal material (such as stainless steel or aluminum) or a polymer material (such as silicone).

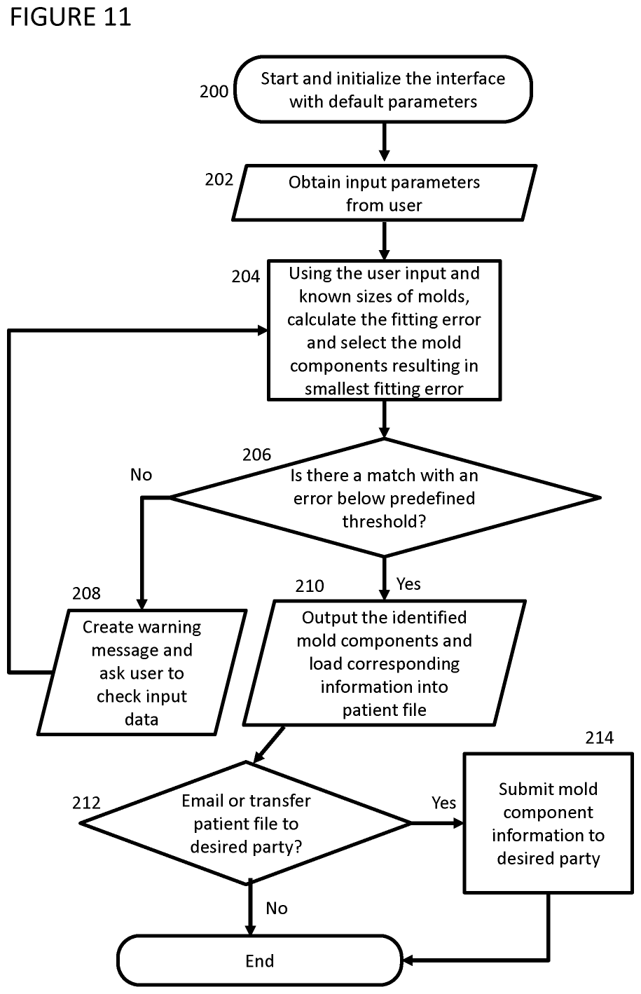

Selection of a three component mold from a library of a plurality of categorized and annotated mold components may be achieved through a computer-implemented method. FIG. 11 shows an illustrative computer-implemented method that automates a user request for a specific three component mold selection with prompts for the user to input size and/or shape parameters of a revision surgery, selects a suitable three component mold combination and return the details of the selected three component mold combination to the user. For example, the computer-implemented method can start by defining variables and populating the variable with default parameters 200 retrieved from memory. The user can then be prompted to provide input parameters 202 of a revision surgery through user interface software. Alternatively, the user may upload a data file formatted to supply input parameters. A processor can then execute a matching algorithm to determine a suitable fit of a three component mold combination to user input parameters. The matching algorithm can include calculation of a fitting error for user input parameters to known sizes of mold components categorized and annotated in a digital library, and selecting a three component mold combination having the smallest fitting error 204. The fitting error of the selected three component mold combination is compared to a predetermined threshold 206. If the fitting error is greater than or equal to the predetermined threshold a warning message is generated and sent to the user 208 with a prompt to check and/or resubmit the input parameters. If the fitting error is less than the predetermined threshold, then identifiers and information of the selected three component mold are stored in a patient file 210. If the user request specifies transfer of the patient file 212 to the user or any desired party, then the patient file is transferred as instructed 214.

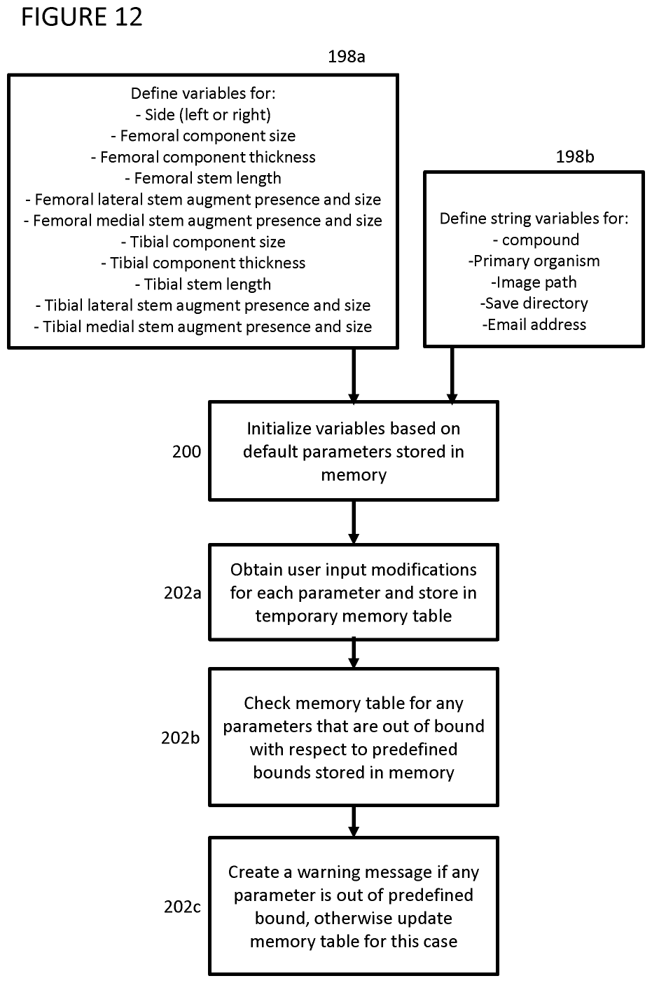

FIG. 12 shows an example of further detailed steps of initializing variables 200 and populating variable with user input parameters 202. Sufficient variables are defined 198a to achieve an efficient and accurate comparison of input parameters to geometries of mold components stored in memory. Any number of variables may be used including any suitable combination from the following group: anatomical side (left or right side); femoral component size, thickness (as described above femoral thickness is typically not adjusted), stem length, whether augments are needed on each side, and if so what size; and tibial component size, thickness, stem length, whether augments are needed on each side, and if so what size. Variables may also be defined to process additional information 198b relating to a user request, such as cement or antibiotic compounds, infectious organism, file storage location, contact information or any other variable considered useful for execution of the computer-implemented method. Variables are initialized with default parameters retrieved from memory 200. User input parameters are retrieved and stored in a temporary memory table 202a. User input parameters are compared against default parameters to determine whether any user input parameters exceed a predetermined threshold 202b, and if so a warning message is generated and sent to the user 202c. If the input parameter remains safely within a predetermined threshold a memory table is updated for use in a matching algorithm.

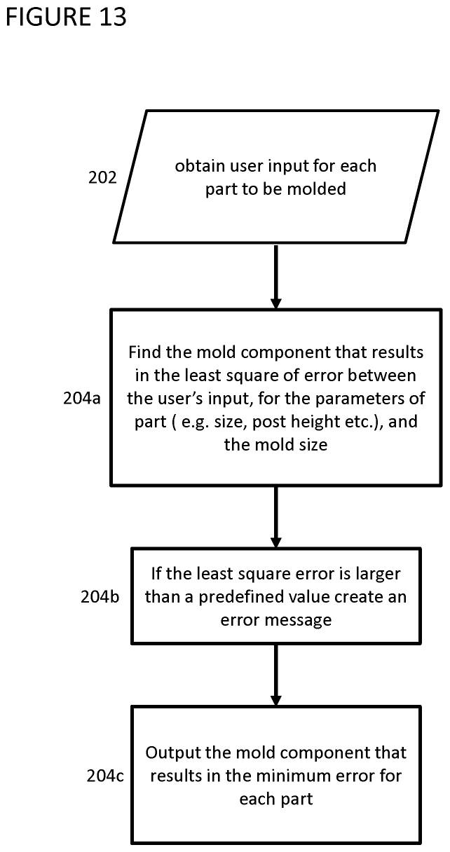

FIG. 13 shows an example of further detailed steps of processing a matching algorithm 204 based on user input parameters and categorized and mold geometries stored in memory. User input parameters are compared against corresponding parameters for a plurality of mold component geometries stored in memory 204a and the least square of error between user input parameters and corresponding mold component parameters are identified and selected. If the least square error is greater than a predetermined threshold than a warning message is generated and communicated to the user 204b. If the least square error is within acceptable limits (less than predetermined threshold) then the mold component is selected and the mold component information is stored in the patient file 204c.

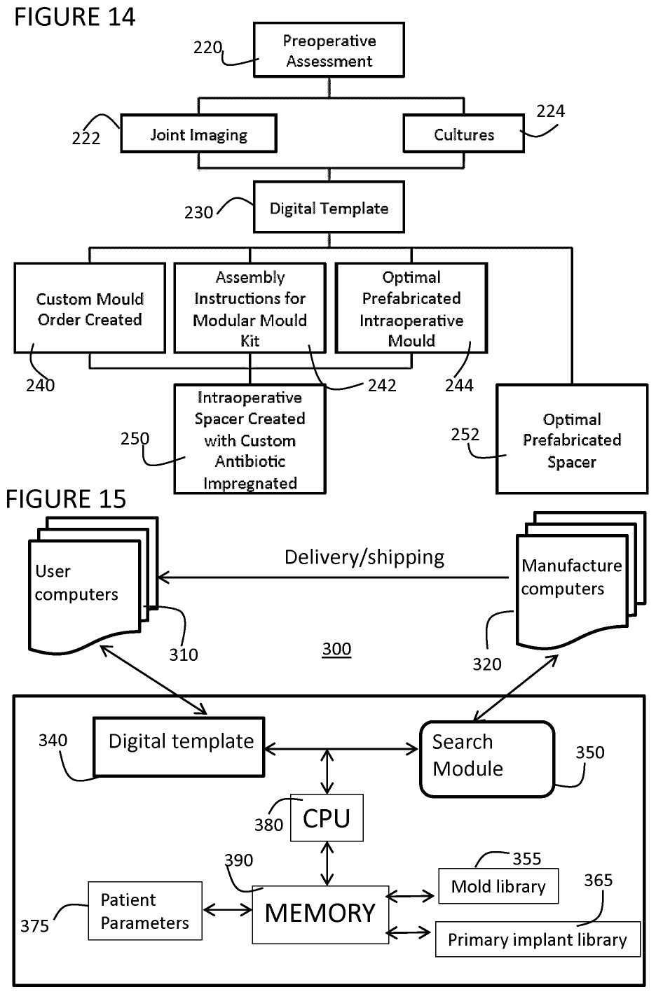

FIG. 14 shows a larger sequence of steps for creating a customized orthopaedic joint spacer encompassing steps shown in FIG. 11. Steps shown in FIG. 11 function to establish a digital template for each patient and to select a three component mold combination from a library containing multiple versions of each component, and possibly multiple sets of versions for one or more of the three components. In FIG. 14, establishing a digital template 230 is shown in the context of examples of input parameters and examples of outcomes subsequent to selection of a three component mold combination from a library.

FIG. 14 illustrates a method that establishes the digital template 230 capable of displaying a patient's pre-operative images and offering the input of a number of patient-specific variables to ultimately output a suitable option for creation of a customized spacer. Output options include for example, a prefabricated spacer/mold, an order for a disposable intraoperative mold or assembly instructions to compose a mold from an existing modular mold kit.

A method for creation of a customized orthopaedic joint spacer as shown for example in FIG. 14 involves populating a digital template with patient specific input parameters to define patient specific variables. Patient specific variables include, for example: primary/permanent implant model, spacer model, component articular surface, component size, component width, component length, component depth, stem size, stem width, stem length, stem depth, surface feature(s), medial anatomical defect, lateral anatomical defect, antibiotic choice, antibiotic dose, or any suitable combination thereof.

Input parameters to define patient specific variables are determined during preoperative assessment 220. Determination of infecting pathogen is typically based upon preoperative joint aspiration and lab cultures 224. Preoperative joint images 222 may be obtained which, when confirmed with patient history/chart, can be used to determine the existing implant model and component size. Images can also be used to assess the potential for anatomical defects.

The preoperative data will be used as patient-specific input variables for a digital template 230 that will execute a matching algorithm to interrogate a library of mold components to determine an optimal combination of mold components to produce a spacer for the patient.

The output of the digital template 230 can provide the user with one or more of various outcomes based upon user preference, as shown in FIG. 14. For example, in one embodiment, the user may choose to be provided an assembly protocol to create an intraoperative mold from a reusable modular mold kit that remains on-site with the user (eg., kit pre-purchased by user). The reusable modular kit include a plurality of first mold components, a plurality of second mold components and a plurality of second mold components with the components of the kit configured to be interchangeably attachable in any combination of first, second and third mold components. Optionally, each components may be marked with a unique indicia or a unique identifier that corresponds to a corresponding identifier listed in a record of the mold components in a mold library. In another embodiment, the output may be the optimal prefabricated spacer available on the market based upon geographic location 252. In another embodiment, the output may be the optimal spacer mould available on the market 244. In another embodiment, the output may be a custom order placed with an appropriate service for the production of sterile, medical-grade, disposable mold for intraoperative use 240. In this example, the result of the possible outputs is either intraoperative production of a customized spacer 250 or delivery of a prefabricated spacer 252 matching the patient specific input parameters.

FIG. 15 shows an example of a computer system graph depict computer architecture communication to implement the illustrative methods shown in FIGS. 11 to 14. The system may implement methods using a single computer, but more often the system will be geographically distributed across multiple computer devices connected through one or more networks. Double ended arrows indicate bidirectional communication and may be mediated through a network. A single arrow indicates an optional shipping of physical components. When configured as networked computer architecture, the system 300 allows multiple end user computers 310 to allow a plurality of individual user to each independently access the computer-implemented methods shown in FIGS. 11 to 12 executed on one or more remote interface servers with communication between end user computers and remote interface server(s) 380 being maintained through a network, such as the Internet. Remote interface server(s) 380 maintain software modules that provide a user interface to input patient specific parameters to establish a digital template 340 as well as providing a search module 350 that executes a matching algorithm to search records of a mold library 355 using patient specific input parameters entered through digital template module 340. Both digital template module 340 and search module 350 may be maintained on one or more servers communicating through networked connections, such as an intranet. The remote interface server(s) operably communicate with one or more memory servers 390 to store digital template 340 data in patient specific files in a patient parameters database 375. The memory server(s) 390 are operably communicative with devices that maintain and/or update records of the patient parameters database 375 as well as maintain and/or update records of the mold library database 355 and optionally maintain and/or update records of the primary implant library database 365. The primary implant library database 365 can include file for each make/model/size of primary implant in a format that corresponds to variables defined in the digital template module 340 so as to easily retrieve primary implant geometries/dimensions to be used by search module 350 for interrogation of mold library database 355. Patient parameters database 375 and mold library database 355 and primary implant library database may be independently maintained on one or more servers communicating through networked connections, such as an intranet. While FIG. 15 is illustrated in a distributed fashion, all software may be consolidated in a single geographical location as desired for a specific implementation. Optionally, the interface server(s) 380 (or the digital template module 340 or the search module 350 as desired for a convenient configuration) may communicate with manufacturer computer devices 320 to send work orders for existing molds or spacers or to send data files for de novo manufacturing. For new manufacturing remote server(s) 380 may be operably communicative with computerized manufacturing machines such as 3D printers or computer controlled milling machines.

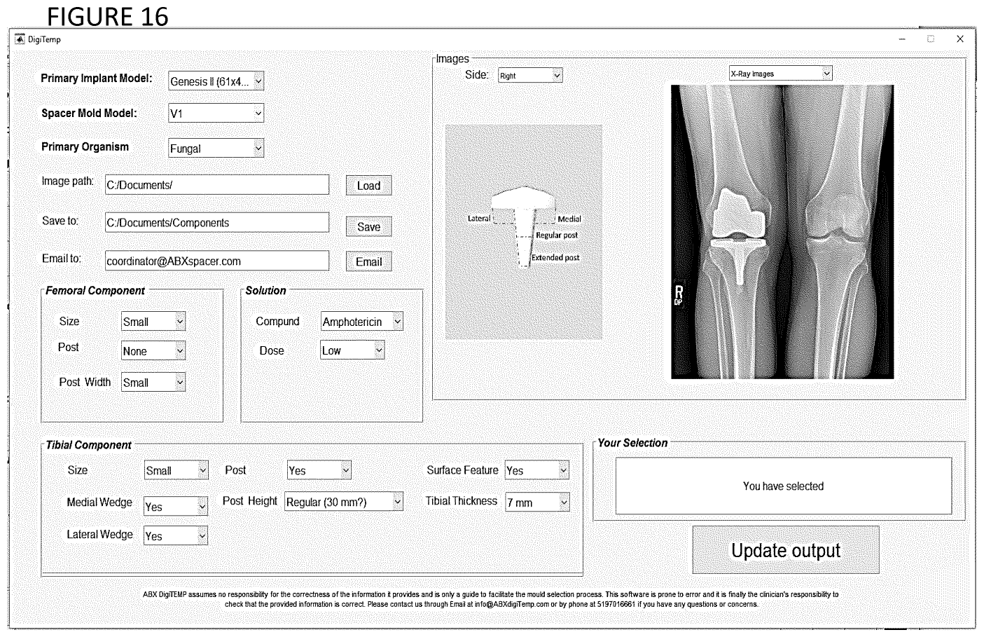

FIG. 16 shows an example of a user graphical interface that may be displayed on a screen/monitor of an end user computer, the graphical user interface providing graphical images, data input boxes and prompts for the user to conveniently input patient specific parameters to define patient specific variables to establish a patient specific digital template. Data input may be through any convenient tool, such as pull-down menu lists or radio button lists. Graphical elements may be positioned and organized as desired to provide users with a convenient interface to establish a patient specific digital template. As shown in FIG. 16, a user interface can display preoperative images and a rendering of a customized spacer (with the ability to select a specific side) as the user inputs variable patient-specific data. The primary implant model, spacer model, and primary infecting organism (based upon preoperative laboratory evidence) may be selected. The antibiotic choice and dose may be selected based upon user preference. Patient-specific variables may also be specified that will contribute to the overall shape, configuration and features of the selected bone cement mold combination and consequently the overall shape, configuration and surface features of the spacer end product.

Embodiments disclosed herein, or portions thereof, can be implemented by programming one or more computer systems or devices with computer-executable instructions embodied in a non-transitory computer-readable medium. When executed by a processor, these instructions operate to cause these computer systems and devices to perform one or more functions particular to embodiments disclosed herein. Programming techniques, computer languages, devices, and computer-readable media necessary to accomplish this are known in the art.

The computer readable medium is a data storage device that can store data, which can thereafter, be read by a computer system. Examples of a computer readable medium include read-only memory, random-access memory, CD-ROMs, magnetic tape, optical data storage devices and the like. The computer readable medium may be geographically localized or may be distributed over a network coupled computer system so that the computer readable code is stored and executed in a distributed fashion.

Computer-implementation of the system or method typically comprises a memory, an interface and a processor. The types and arrangements of memory, interface and processor may be varied according to implementations. For example, the interface may include a software interface that communicates with an end-user computing device through an Internet connection. The interface may also include a physical electronic device configured to receive requests or queries from an end-user.

Any suitable processor type may be used depending on a specific implementation, including for example, a microprocessor, a programmable logic controller or a field programmable logic array. Moreover, any conventional computer architecture may be used for computer-implementation of the system or method including for example a memory, a mass storage device, a processor (CPU), a Read-Only Memory (ROM), and a Random-Access Memory (RAM) generally connected to a system bus of data-processing apparatus. Memory can be implemented as a ROM, RAM, a combination thereof, or simply a general memory unit. Software modules in the form of routines and/or subroutines for carrying out features of the system or method can be stored within memory and then retrieved and processed via processor to perform a particular task or function. Similarly, one or more method steps may be encoded as a program component, stored as executable instructions within memory and then retrieved and processed via a processor. A user input device, such as a keyboard, mouse, or another pointing device, can be connected to PCI (Peripheral Component Interconnect) bus. If desired, the software may provide an environment that represents programs, files, options, and so forth by means of graphically displayed icons, menus, and dialog boxes on a computer monitor screen.

Computer-implementation of the system or method may accommodate any type of end-user computing device including computing devices communicating over a networked connection. The computing device may display graphical interface elements for performing the various functions of the system or method. For example, the computing device may be a server, desktop, laptop, notebook, tablet, personal digital assistant (PDA), PDA phone or smartphone, and the like. The computing device may be implemented using any appropriate combination of hardware and/or software configured for wired and/or wireless communication. Communication can occur over a network, for example, where remote control of the system is desired.

If a networked connection is desired the system or method may accommodate any type of network. The network may be a single network or a combination of multiple networks. For example, the network may include the internet and/or one or more intranets, landline networks, wireless networks, and/or other appropriate types of communication networks. In another example, the network may comprise a wireless telecommunications network (e.g., cellular phone network) adapted to communicate with other communication networks, such as the Internet. For example, the network may comprise a computer network that makes use of a TCP/IP protocol (including protocols based on TCP/IP protocol, such as HTTP, HTTPS or FTP).

Embodiments described herein are intended for illustrative purposes without any intended loss of generality. Still further variants, modifications and combinations thereof are contemplated and will be recognized by the person of skill in the art. Accordingly, the foregoing detailed description is not intended to limit scope, applicability, or configuration of claimed subject matter.

1.-40. (canceled)

41. A modular bone cement mold kit for producing a bone cement spacer having a head portion integrally connected to a stem portion, the kit comprising:
a set of a plurality of versions of a first mold component, each of the plurality of versions of the first mold component defining a first cavity shaped to form at least a majority of the head portion of the spacer;
a set of a plurality of versions of a second mold component, each of the plurality of versions of the second mold component defining a second cavity shaped to form a first part of a stem portion of the spacer and at least one of the plurality of versions of the second mold component defining an augmented surface feature at or near a base end of the first part of the stem portion;
a set of a plurality of versions of a third mold component, each of the plurality of versions of the third mold component defining a third cavity shaped to form a second part of the stem portion of the spacer and at least one of the plurality of versions of the third mold component defining an augmented surface feature at or near the base end of the second part of the stem portion;
instructions for matching of a single version from each set to a patient specific parameter to select a matched three-component mold comprising a matched first mold component, a matched second mold component, and a matched third mold component;
an abutting combination of the matched first mold component, the matched second mold component, and the matched third component defining a communicative cavity shaped to form the head portion integrally connected with the stem portion of the spacer, the matched second mold component and the matched third mold component combining to define a cavity shaped to form at least a majority of the stem portion.

42. The kit of claim 41, wherein the first cavity in each of the plurality of versions of the first mold component defines an articulating surface and a sidewall of the head portion.

43. The kit of claim 42, wherein the matched second mold component and the matched third mold component combine to define a surface shaped to form a bone-facing surface of the head portion.

44. The kit of claim 41, wherein the second cavity and the third cavity are symmetrically shaped.

45. The kit of claim 41, wherein the second cavity and the third cavity are asymmetrically shaped.

46. The kit of claim 41, wherein each of the plurality of versions of the second component defines a different size of augmented surface feature and each of the plurality of versions of the third component defines a different size of augmented surface feature.

47. The kit of claim 41, further comprising a handle formed on at least one of the first, second or third mold components.

48. The kit of claim 41, further comprising an inlet port formed in at least one of the first, second or third mold components.

49. The kit of claim 41, further comprising a ventilation aperture formed in at least one of the first, second or third mold components.

50. The kit of claim 41, further comprising an endoskeleton disposed within the communicative cavity formed from the abutting combination of the matched first, second and third mold components.

51. The kit of claim 41, wherein the communicative cavity is shaped to form a knee joint spacer or a hip joint spacer or a shoulder joint spacer.

52. The kit of claim 51, wherein the knee joint spacer is a femoral spacer or a tibial spacer.

53. The kit of claim 41, further comprising one or more of bone cement powder, a polymerization initiator, an endoskeleton, or a bone cement applicator.

54. The kit of claim 41, wherein the instructions comprise a table for assisting selection of the matched three-component mold.

55. A system for selecting a bone cement mold, the system comprising:
the kit of claim 41;
a memory configured to store records of a mold digital library, the mold library including records of dimensions of the plurality of versions of the first mold component, the plurality of versions of the second mold component, and the plurality of versions of the third mold component;
an interface device connected to a network, the interface device configured to receive a request for a bone cement mold combination, the request including the patient specific parameter relating to a joint surgery;
a processor operably communicative with the interface device, the processor configured to use the patient specific parameter to search the mold library stored in memory, and select the matched first mold component, the matched second mold component and the matched third mold component that combine to form the matched three-component mold that matches the patient specific parameter.

56. The system of claim 55, wherein the patient specific parameter is stored in the memory.

57. The system of claim 55, further comprising a primary implant library stored in the memory, the primary implant library including records of dimensions of a plurality of primary implants used in joint surgery.

58. The system of claim 55, wherein the interface device maintains a graphical user interface to prompt an input of the patient specific parameter.

59. The system of claim 55, wherein the processor executes a matching algorithm using the patient specific parameter to search the mold library.

60. The system of claim 59, wherein the matching algorithm includes a best fit or least error comparison of a plurality of dimensions of each mold component with a corresponding plurality of the patient specific parameter.
